SEQUENCE LISTING (4 sequences)

SEQ ID NO: 1 - protein, 107 aa, Artificial sequence
  Sequence is synthesized.
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 2 - protein, 122 aa, Artificial sequence [sequence omitted]

SEQ ID NO: 3 - protein, 232 aa, Artificial sequence
  Sequence is synthesized.
MGWSCIILFLVATATGVHSDIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 471 aa, Artificial sequence
  Sequence is synthesized.
MGWSCIILFLVATATGVHSEVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2005/0032130 A1
(43) Pub. Date: Feb. 10, 2005

(54) Neutralizing antibody assay and uses therefor

(75) Inventors: Maureen Beresini, Moss Beach, CA (US); An Song, Palo Alto, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 10/877,363

(22) Filed: Jun. 24, 2004

Related U.S. Application Data
(60) Provisional application No. 60/490,678, filed on Jul. 29, 2003.

(51) Int. Cl. G01N 33/53; G01N 33/567

(52) U.S. Cl. 435/7.2

(57) ABSTRACT

A method of detecting neutralizing antibodies to a therapeutic antibody such as a CD20 antibody is described. The assay can be used to determine the efficacy of the antibody in a method of immunotherapy.

This is a non-provisional application claiming priority under 35 USC §119 to provisional application No. 60/490,678 filed Jul. 29, 2003, the entire disclosure of which is hereby incorporated by reference.

### FIELD OF THE INVENTION

The present invention concerns an assay for detecting neutralizing antibodies against an antibody or antagonist, and uses for that assay.

### BACKGROUND OF THE INVENTION

Lymphocytes are one of many types of white blood cells produced in the bone marrow during the process of hematopoiesis. There are two major populations of lymphocytes: B lymphocytes (B cells) and T lymphocytes (T cells). The lymphocytes of particular interest herein are B cells.

B cells mature within the bone marrow and leave the marrow expressing an antigen-binding antibody on their cell surface. When a naive B cell first encounters the antigen for which its membrane-bound antibody is specific, the cell begins to divide rapidly and its progeny differentiate into memory B cells and effector cells called “plasma cells”. Memory B cells have a longer life span and continue to express membrane-bound antibody with the same specificity as the original parent cell. Plasma cells do not produce membrane-bound antibody but instead produce the antibody in a form that can be secreted. Secreted antibodies are the major effector molecule of humoral immunity.

The CD20 antigen (also called human B-lymphocyte-restricted differentiation antigen, Bp35) is a hydrophobic transmembrane protein with a molecular weight of approximately 35 kD located on pre-B and mature B lymphocytes (Valentine et al. J. Biol. Chem. 264(19):11282-11287 (1989); and Einfeld et al. EMBO J. 7(3):711-717 (1988)). The antigen is also expressed on greater than 90% of B cell non-Hodgkin's lymphomas (NHL) (Anderson et al. Blood 63(6):1424-1433 (1984)), but is not found on hematopoietic stem cells, pro-B cells, normal plasma cells or other normal tissues (Tedder et al. J. Immunol. 135(2):973-979 (1985)). CD20 regulates an early step(s) in the activation process for cell cycle initiation and differentiation (Tedder et al., supra) and possibly functions as a calcium ion channel (Tedder et al. J. Cell. Biochem. 14D:195 (1990)).

Given the expression of CD20 in B cell lymphomas, this antigen can serve as a candidate for “targeting” of such lymphomas. In essence, such targeting can be generalized as follows: antibodies specific to the CD20 surface antigen of B cells are administered to a patient. These anti-CD20 antibodies specifically bind to the CD20 antigen of (ostensibly) both normal and malignant B cells; the antibody bound to the CD20 surface antigen may lead to the destruction and depletion of neoplastic B cells. Additionally, chemical agents or radioactive labels having the potential to destroy the tumor can be conjugated to the anti-CD20 antibody such that the agent is specifically “delivered” to the neoplastic B cells. Irrespective of the approach, a primary goal is to destroy the tumor; the specific approach can be determined by the particular anti-CD20 antibody which is utilized and, thus, the available approaches to targeting the CD20 antigen can vary considerably.

CD19 is another antigen that is expressed on the surface of cells of the B lineage. Like CD20, CD19 is found on cells throughout differentiation of the lineage from the stem cell stage up to a point just prior to terminal differentiation into plasma cells (Nadler, L. Lymphocyte Typing II 2: 3-37 and Appendix, Renling et al. eds. (1986) by Springer Verlag). Unlike CD20 however, antibody binding to CD19 causes internalization of the CD19 antigen. CD19 antigen is identified by the HD237-CD19 antibody (also called the “B4” antibody) (Kiesel et al. Leukemia Research II, 12: 1119 (1987)), among others. The CD19 antigen is present on 4-8% of peripheral blood mononuclear cells and on greater than 90% of B cells isolated from peripheral blood, spleen, lymph node or tonsil. CD19 is not detected on peripheral blood T cells, monocytes or granulocytes. Virtually all non-T cell acute lymphoblastic leukemias (ALL), B cell chronic lymphocytic leukemias (CLL) and B cell lymphomas express CD19 detectable by the antibody B4 (Nadler et al. J. Immunol. 131:244 (1983); and Nadler et al. in Progress in Hematology Vol. XII pp. 187-206. Brown, E. ed. (1981) by Grune & Stratton, Inc).

Additional antibodies which recognize differentiation stage-specific antigens expressed by cells of the B cell lineage have been identified. Among these are the B2 antibody directed against the CD21 antigen; B3 antibody directed against the CD22 antigen; and the J5 antibody directed against the CD10 antigen (also called CALLA). See U.S. Pat. No. 5,595,721 issued Jan. 21, 1997 (Kaminski et al.).

The rituximab (RITUXAN®) antibody is a genetically engineered chimeric murine/human monoclonal antibody directed against the CD20 antigen. Rituximab is the antibody called “C2B8” in U.S. Pat. No. 5,736,137 issued Apr. 7, 1998 (Anderson et al.). RITUXAN® is indicated for the treatment of patients with relapsed or refractory low-grade or follicular, CD20 positive, B cell non-Hodgkin's lymphoma. In vitro mechanism of action studies have demonstrated that RITUXAN® binds human complement and lyses lymphoid B cell lines through complement-dependent cytotoxicity (CDC) (Reff et al. Blood 83(2):435-445 (1994)). Additionally, it has significant activity in assays for antibody-dependent cell-mediated cytotoxicity (ADCC). More recently, RITUXAN® has been shown to have anti-proliferative effects in tritiated thymidine incorporation assays and to induce apoptosis directly, while other anti-CD19 and CD20 antibodies do not (Maloney et al. Blood 88(10):637a (1996)). Synergy between RITUXAN® and chemotherapies and toxins has also been observed experimentally. In particular, RITUXAN® sensitizes drug-resistant human B cell lymphoma cell lines to the cytotoxic effects of doxorubicin, CDDP, VP-16, diphtheria toxin and ricin (Demidem et al. Cancer Chemotherapy & Radiopharmaceuticals 12(3):177-186 (1997)). In vivo preclinical studies have shown that RIFUXAN® depletes B cells from the peripheral blood, lymph nodes, and bone marrow of cynomolgus monkeys, presumably through complement and cell-mediated processes (Reff et al. Blood 83(2):435-445 (1994)).

Patents and patent publications concerning CD20 antibodies include U.S. Pat. Nos. 5,776,456, 5,736,137, 6,399,061, and 5,843,439, as well as U.S. patent appln Nos. US 2002/0197255A1 and US 2003/0021781A1 (Anderson et al.); U.S. Pat. No. 6,455,043B1 and WO00/09160 (Grillo-Lopez, A.); WO00/27428 (Grillo-Lopez and White); WO00/27433 (Grillo-Lopez and Leonard); WO00/44788 (Braslawsky et al.); WO01/10462 (Rastetter, W.); WO01/10461 (Rastetter and White); WO01/10460 (White and Grillo-Lopez); U.S. appln No. US2002/0006404 and WO02/04021 (Hanna and Hariharan); U.S. appln No. US2002/0012665 A1 and WO01/74388 (Hanna, N.); U.S. appln No. US2002/0009444A1, and WO01/80884 (Grillo-Lopez, A.); WO01/97858 (White, C.); U.S. appln No. US2002/0128488A1 and WO02/34790 (Reff, M.);WO02/060955 (Braslawsky et al.);WO2/096948 (Braslawsky et al.);WO02/079255 (Reff and Davies); U.S. Pat. No. 6,171,586B1, and WO98/56418 (Lam et al.); WO98/58964 (Raju, S.); WO99/22764 (Raju, S.);WO99/51642, U.S. Pat. No. 6,194,551B1, U.S. Pat. No. 6,242,195B1, U.S. Pat. No. 6,528,624B1 and U.S. Pat. No. 6,538,124 (Idusogie et al.); WO00/42072 (Presta, L.); WO00/67796 (Curd et al.); WO01/03734 (Grillo-Lopez et al.); U.S. appln No. US 2002/0004587A1 and WO01/77342 (Miller and Presta); U.S. appln No. US2002/0197256 (Grewal, I.); U.S. Pat. Nos. 6,090,365B1, 6,287,537B1, 6,015,542, 5,843,398, and 5,595,721, (Kaminski et al.); U.S. Pat. Nos. 5,500,362, 5,677,180, 5,721,108, and 6,120,767 (Robinson et al.); U.S. Pat No. 6,410,391B1 (Raubitschek et al.); U.S. Pat. No. 6,224,866B1 and WO00/20864 (Barbera-Guillem, E.); WO01/13945 (Barbera-Guillem, E.); WO00/67795 (Goldenberg); WO00/74718 (Goldenberg and Hansen); WO00/76542 (Golay et al.);WO01/72333 (Wolin and Rosenblatt); U.S. Pat. No. 6,368,596B 1 (Ghetie et al.); U.S. Appln No. US2002/0041847A1, (Goldenberg, D.); U.S. Appln No. US2003/0026801A1 (Weiner and Hartmann); WO02/102312 (Engleman, E.), each of which is expressly incorporated herein by reference. See, also, U.S. Pat. No. 5,849,898 and EP appln No. 330,191 (Seed et al.); U.S. Pat. No. 4,861,579 and EP332,865A2 (Meyer and Weiss); and WO95/03770 (Bhat et al.).

Publications concerning therapy with Rituximab include: Perotta and Abuel “Response of chronic relapsing ITP of 10 years duration to Rituximab” Abstract #3360 Blood 10(1)(part 1-2): p. 88B (1998); Stashi et al. “Rituximab chimeric anti-CD20 monoclonal antibody treatment for adults with chronic idopathic thrombocytopenic purpura” Blood 98(4):952-957 (2001); Matthews, R. “Medical Heretics” New Scientist (7 Apr., 2001); Leandro et al. “Clinical outcome in 22 patients with rheumatoid arthritis treated with B lymphocyte depletion” Ann Rheum Dis 61:833-888 (2002); Leandro et al. “Lymphocyte depletion in rheumatoid arthritis: early evidence for safety, efficacy and dose response. Arthritis and Rheumatism 44(9): S370 (2001); Leandro et al. “An open study of B lymphocyte depletion in systemic lupus erythematosus”, Arthritis & Rheumatism 46(1):2673-2677 (2002); Edwards and Cambridge “Sustained improvement in rheumatoid arthritis following a protocol designed to deplete B lymphocytes” Rhematology 40:205-211 (2001); Edwards et al. “B-lymphocyte depletion therapy in rheumatoid arthritis and other autoimmune disorders” Biochem. Soc. Trans. 30(4):824-828 (2002); Edwards et al. “Efficacy and safety of Rituximab, a B-cell targeted chimeric monoclonal antibody: A randomized, placebo controlled trial in patients with rheumatoid arthritis. Arthritis and Rheumatism 46(9): S197 (2002); Levine and Pestronk “IgM antibody-related polyneuropathies: B-cell depletion chemotherapy using Rituximab” Neurology 52: 1701-1704 (1999); DeVita et al. “Efficacy of selective B cell blockade in the treatment of rheumatoid arthitis” Arthritis & Rheum 46:2029-2033 (2002); Hidashida et al. “Treatment of DMARD-Refractory rheumatoid arthritis with rituximab.” Presented at the Annual Scientific Meeting of the American College of Rheumatology; October 24-29; New Orleans, La. 2002; Tuscano, J. “Successful treatment of Infliximab-refractory rheumatoid arthritis with rituximab” Presented at the Annual Scientific Meeting of the American College of Rheumatology; October 24-29; New Orleans, La. 2002.

U.S. patent application No. 2003/0068664 (Albitar et al.) describes an ELISA assay for determining human anti-chimeric antibody (HACA) directed against Rituximab.

### SUMMARY OF THE INVENTION

Example 1 herein describes the development of a complement-dependent cytotoxicity (CDC) assay for detecting neutralizing antibodies against an antibody that binds a B cell surface marker, namely the CD20 antigen. The CDC activity was measured by incubating CD20 positive cells with human complement in the absence or presence of different concentrations of the CD20 antibody. Cytotoxicity was then measured by quantifying live cells. Serum matrix effect on assay performance was tested. Serum could be tolerated up to 40% without a significant shift in EC50 values. CD20 antibody-treated systemic lupus erythrematosis (SLE) patient serum samples with an antibody response (HACA) were then tested. The CDC activity of the CD20 antibody could be either completely or partially blocked with HACA sera, indicating neutralizing activities in treated samples. In comparison, serum samples obtained prior to CD20 antibody treatment showed no neutralizing activity. This assay characterizes the nature of any anti-drug antibody response; therefore it will be valuable for evaluating drug safety and efficacy.

Accordingly, the present invention provides a method for evaluating the efficacy of an antibody that binds CD20 comprising measuring the ability of a biological sample from a patient treated with the CD20 antibody to block a biological activity of the CD20 antibody.

The invention further provides a method of immunotherapy comprising administering an antibody that binds CD20 to a patient; and measuring the ability of a biological sample from the patient to block a biological activity of the CD20 antibody.

In another aspect, the invention concerns a method of detecting neutralizing antibodies to a therapeutic antibody comprising exposing cells that express an antigen to which the therapeutic antibody binds to complement in the presence of the therapeutic antibody and a biological sample from a patient treated therewith; and determining complement-dependent cytotoxicity (CDC) activity of the therapeutic antibody, wherein a reduction in the CDC activity indicates the presence of neutralizing antibodies in the biological sample.

Additionally, a method of evaluating the efficacy of an antagonist that binds a B cell surface marker is provided which comprises measuring the ability of a biological sample from a patient treated with the antagonist to block a biological activity of the antagonist.

In yet a further embodiment, the invention concerns a method of immunotherapy comprising

administering an antibody that binds a B cell surface marker to a patient; and measuring the ability of a biological sample from the patient to block a biological activity of the antibody.

### DETAILED DESCRIPTION OF THE PREFERRED EMBODIMENTS

I. Definitions

Unless indicated otherwise, by “biological sample” herein is meant a sample obtained from a patient herein. The sample may comprise antibodies that bind to the antibody or drug with which the patient has been treated, such as human anti-murine antibody (HAMA), human anti-chimeric antibody (HACA) or human anti-human antibody (HAHA). The biological sample may for example be serum, antibodies recovered from the patient, plasma, cell lysate, milk, saliva, and other secretions, but preferably serum.

The expression “biological activity” refers to a measurable function of an antibody or antagonist herein. Various activities are contemplated and include, but are not limited to, complement-dependent cytotoxicity (CDC), antibody-dependent cell-mediated cytotoxicity (ADCC), apoptosis, inhibiting growth of cells (e.g. tumor cells), etc.

The ability of a biological sample (or antibodies raised by a patient against the drug in question) to “block” a biological activity of an antagonist or antibody refers to both partial and complete blocking of that activity.

A “B cell surface marker” herein is an antigen expressed on the surface of a B cell which can be targeted with an antagonist or antibody which binds thereto. Exemplary B cell surface markers include the CD 10, CD 19, CD20, CD21, CD22, CD23, CD24, CD37, CD40, CD53, CD72, CD73, CD74, CDw75, CDw76, CD77, CDw78, CD79a, CD79b, CD80, CD81, CD82, CD83, CDw84, CD85 and CD86 leukocyte surface markers. The B cell surface marker of particular interest is preferentially expressed on B cells compared to other non-B cell tissues of a mammal and may be expressed on both precursor B cells and mature B cells. In one embodiment, the marker is one, like CD20 or CD 19, which is found on B cells throughout differentiation of the lineage from the stem cell stage up to a point just prior to terminal differentiation into plasma cells. The preferred B cell surface marker herein is CD20.

The “CD20” antigen is a ˜35 kDa, non-glycosylated phosphoprotein found on the surface of greater than 90% of B cells from peripheral blood or lymphoid organs. CD20 is expressed during early pre-B cell development and remains until plasma cell differentiation. CD20 is present on both normal B cells as well as malignant B cells. Other names for CD20 in the literature include “B-lymphocyte-restricted antigen” and “Bp35”. The CD20 antigen is described in Clark et al. PNAS (USA) 82:1766 (1985), for example.

As used herein, “B cell depletion” refers to a reduction in B cell levels in an animal or human generally after drug or antibody treatment, as compared to the level before treatment. B cell depletion can be partial or complete. B cell levels are measurable using well known techniques such as those described in Reff et al., Blood 83: 435-445 (1994), or U.S. Pat. No. 5,736,137 (Anderson et al.). By way of example, a mammal (e.g. a normal primate) may be treated with various dosages of the antibody or antagonist, and peripheral B-cell concentrations may be determined, e.g. by a FACS method that counts B cells.

A “B cell malignancy” is a malignancy involving B cells. Examples include Hodgkin's disease, including lymphocyte predominant Hodgkin's disease (LPHD); non-Hodgkin's lymphoma (NHL); follicular center cell (FCC) lymphoma; acute lymphocytic leukemia (ALL); chronic lymphocytic leukemia (CLL); hairy cell leukemia; plasmacytoid lymphocytic lymphoma; mantle cell lymphoma; AIDS or HIV-related lymphoma; multiple myeloma; central nervous system (CNS) lymphoma; post-transplant lymphoproliferative disorder (PTLD); Waldenstrom's macroglobulinemia (lymphoplasmacytic lymphoma); mucosa-associated lymphoid tissue (MALT) lymphoma; and marginal zone lymphoma/leukemia.

Non-Hodgkin's lymphoma (NHL) includes, but is not limited to, low grade/follicular NHL, relapsed or refractory NHL, front line low grade NHL, Stage III/IV NHL, chemotherapy resistant NHL, small lymphocytic (SL) NHL, intermediate grade/follicular NHL, intermediate grade diffuse NHL, diffuse large cell lymphoma, aggressive NHL (including aggressive front-line NHL and aggressive relapsed NHL), NHL relapsing after or refractory to autologous stem cell transplantation, high grade immunoblastic NHL, high grade lymphoblastic NHL, high grade small non-cleaved cell NHL, bulky disease NHL, etc.

An “autoimmune disease” herein is a disease or disorder arising from and directed against an individual's own tissues. Examples of autoimmune diseases or disorders include, but are not limited to arthritis (rheumatoid arthritis, juvenile rheumatoid arthritis, osteoarthritis, psoriatic arthritis), psoriasis, dermatitis, polymyositis/dermatomyositis, toxic epidermal necrolysis, systemic scleroderma and sclerosis, responses associated with inflammatory bowel disease, Crohn's disease, ulcerative colitis, respiratory distress syndrome, adult respiratory distress syndrome (ARDS), meningitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), juvenile onset diabetes, multiple sclerosis, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including ANCA), aplastic anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, central nervous system (CNS) inflammatory disorders, multiple organ injury syndrome, mysathenia gravis, antigen-antibody complex mediated diseases, anti-glomerular basement membrane disease, anti-phospholipid antibody syndrome, allergic neuritis, Bechet disease, Castleman's syndrome, Goodpasture's syndrome, Lambert-Eaton Myasthenic Syndrome, Reynaud's syndrome, Sjogren's syndrome, Stevens-Johnson syndrome, solid organ transplant rejection, graft versus host disease (GVHD), pemphigoid bullous, pemphigus, autoimmune polyendocrinopathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), including fludarabine-associated ITP, thrombotic thrombocytopenic purpura (TTP), autoimmune thrombocytopenia, autoimmune disease of the testis and ovary including autoimune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, lymphoid interstitial pneumonitis (HIV), bronchiolitis obliterans (non-transplant) vs NSIP, Guillain-Barre' syndrome, large vessel vasculitis (including polymyalgia rheumatica and giant cell (Takayasu's) arteritis), medium vessel vasculitis (including Kawasaki's disease and polyarteritis nodosa), ankylosing spondylitis, Berger's disease (IgA nephropathy), rapidly progressive glomerulonephritis, primary biliary cirrhosis, Celiac sprue (gluten enteropathy), cryoglobulinemia, amyotrophic lateral sclerosis (ALS), coronary artery disease, cold agglutinin disease, acquired factor VIII inhibitors, lupus nephritis, etc.

An “antagonist” that binds a B cell surface marker herein is a molecule which, upon binding to a B cell surface marker, destroys or depletes B cells in a mammal and/or interferes with one or more B cell functions, e.g. by reducing or preventing a humoral response elicited by the B cell. The antagonist preferably is able to deplete B cells in a mammal treated therewith. Such depletion may be achieved via various mechanisms such antibody-dependent cell-mediated cytotoxicity (ADCC) and/or complement dependent cytotoxicity (CDC), inhibition of B cell proliferation and/or induction of B cell death (e.g. via apoptosis). Antagonists included within the scope of the present invention include antibodies, synthetic or native sequence peptides, immunoadhesins, small molecule antagonists which bind to the B cell marker, optionally conjugated with or fused to a cytotoxic agent. The preferred antagonist comprises an antibody.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. Preferably, the cells express at least FcγRIII and carry out ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIII (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)). FcRs herein include polymorphisms such as the genetic dimorphism in the gene that encodes FcγRIIIa resulting in either a phenylalanine (F) or a valine (V) at amino acid position 158, located in the region of the receptor that binds to IgG1. The homozygous valine FcγRIIIa (FcγRIIIa-158V) has been shown to have a higher affinity for human IgG1 and mediate increased ADCC in vitro relative to homozygous phenylalanine FcγRIIIa (FcγRIIIa-158F) or heterozygous (FcγRIIIa-158F/V) receptors.

“Complement dependent cytotoxicity” or “CDC” refer to the ability of a molecule to lyse a target in the presence of complement. The complement activation pathway is initiated by the binding of the first component of the complement system (C1q) to a molecule (e.g. an antibody) complexed with a cognate antigen. To assess complement activation, a CDC assay, e.g. as described in Gazzano-Santoro et al., J. Immunol. Methods 202:163 (1996), may be performed.

“Growth inhibitory” -antagonists or antibodies are those which prevent or reduce proliferation of a cell expressing an antigen to which the antagonist binds. For example, the antagonist or antibody may prevent or reduce proliferation of B cells in vitro and/or in vivo.

Antagonists or antibodies which “induce apoptosis” are those which induce programmed cell death, e.g. of a B cell, as may be determined by standard apoptosis assays, such as binding of annexin V, fragmentation of DNA, cell shrinkage, dilation of endoplasmic reticulum, cell fragmentation, and/or formation of membrane vesicles (called apoptotic bodies).

The term “antibody” herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity.

“Antibody fragments” comprise a portion of an intact antibody, preferably comprising the antigen-binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody-dependent cell-mediated cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ∈, γ, and μ, respectively. The subunit structures and three-dimensional configurations of different classes of immunoglobulins are well known.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Phannacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (V_{H}) connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “hypervariable region” when used herein refers to the amino acid residues of an antibody which are responsible for antigen-binding. The hypervariable region comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework” or “FR” residues are those variable domain residues other than the hypervariable region residues as herein defined.

An antagonist or antibody “which binds” an antigen of interest, e.g. a B cell surface marker or CD20, is one capable of binding that antigen with sufficient affinity and/or avidity such that the antagonist or antibody is useful as a therapeutic agent for targeting a cell expressing the antigen.

For the purposes herein, “immunotherapy” will refer to a method of treating a mammal (preferably a human patient) with an antibody, wherein the antibody may be an unconjugated or “naked” antibody, or the antibody may be conjugated or fused with heterologous molecule(s) or agent(s), such as one or more cytotoxic agent(s), thereby generating an “immunoconjugate”.

As used herein, a “therapeutic antibody” is an antibody that is effective in treating a disease or disorder in a mammal with or predisposed to the disease or disorder. Exemplary therapeutic antibodies include anti-HER2 antibodies including rhuMAb 4D5 (HERCEPTIN®) (Carter et al., Proc. Natl. Acad. Sci. USA, 89:4285-4289 (1992), U.S. Pat. No. 5,725,856); anti-CD20 antibodies (see below); anti-IL-8 (St John et al., Chest, 103:932 (1993), and International Publication No. WO 95/23865); anti-VEGF antibodies including humanized and/or affinity matured anti-VEGF antibodies such as the humanized anti-VEGF antibody huA4.6.1 AVASTIN™ (Kim et al., Growth Factors, 7:53-64 (1992), International Publication No. WO 96/30046, and WO 98/45331, published Oct. 15, 1998); anti-PSCA antibodies (WO01/40309); anti-CD40 antibodies, including S2C6 and humanized variants thereof (WO00/75348); anti-CD11a antibodies including Raptiva™ (U.S. Pat. No. 5,622,700, WO 98/23761, Steppe et al., Transplant Intl. 4:3-7 (1991), and Hourmant et al., Transplantation 58:377-380 (1994)); anti-IgE antibodies (Presta et al., J. Immunol. 151:2623-2632 (1993), and International Publication No. WO 95/19181; U.S. Pat. No. 5,714,338, issued Feb. 3, 1998 or U.S. Pat. No. 5,091,313, issued Feb. 25, 1992, WO 93/04173 published Mar. 4, 1993, or International Application No. PCT/US98/13410 filed Jun. 30, 1998, U.S. Pat. No. 5,714,338); anti-CD18 antibodies (U.S. Pat. No. 5,622,700, issued Apr. 22, 1997, or as in WO 97/26912, published Jul. 31, 1997); anti-Apo-2 receptor antibody antibodies (WO 98/51793 published Nov. 19, 1998); anti-TNF-α antibodies including cA2 (REMICADE®), CDP571 and MAK-195 (See, U.S. Pat. No. 5,672,347 issued Sep. 30, 1997, Lorenz et al. J. Immunol. 156(4):1646-1653 (1996), and Dhainaut et al. Crit. Care Med. 23(9):1461-1469 (1995)); anti-Tissue Factor (TF) antibodies (European Patent No. 0 420 937 B1 granted Nov. 9, 1994); anti-human α₄-β₇ integrin antibodies (WO 98/06248 published Feb. 19, 1998); anti-EGFR antibodies (chimerized or humanized 225 antibody as in WO 96/40210 published Dec. 19, 1996); anti-CD3 antibodies such as OKT3 (U.S. Pat. No. 4,515,893 issued May 7, 1985); anti-CD25 or anti-Tac antibodies such as CHI-621 (SIMULECT®) and ZENAPAX® (See U.S. Pat. No. 5,693,762 issued Dec. 2, 1997); anti-CD4 antibodies such as the cM-7412 antibody (Choy et al. Arthritis Rheum 39(1):52-56 (1996)); anti-CD52 antibodies such as CAMPATH-1H (Riechmann et al. Nature 332:323-337 (1988); anti-Fc receptor antibodies such as the M22 antibody directed against FcγRI as in Graziano et al. J. Immunol. 155(10):4996-5002 (1995); anti-carcinoembryonic antigen (CEA) antibodies such as hMN-14 (Sharkey et al. Cancer Res. 55(23Suppl): 5935s-5945s (1995); antibodies directed against breast epithelial cells including huBrE-3, hu-Mc 3 and CHL6 (Ceriani et al. Cancer Res. 55(23): 5852s-5856s (1995); and Richman et al. Cancer Res. 55(23 Supp): 5916s-5920s (1995)); antibodies that bind to colon carcinoma cells such as C242 (Litton et al. Eur J. Immunol. 26(1):1-9 (1996)); anti-CD38 antibodies, e.g. AT 13/5 (Ellis et al. J. Immunol. 155(2):925-937 (1995)); anti-CD33 antibodies such as Hu M195 (Jurcic et al. Cancer Res 55(23 Suppl):5908s-5910s (1995) and CMA-676 or CDP771; anti-CD22 antibodies such as LL2 or LymphoCide (Juweid et al. Cancer Res 55(23 Suppl):5899s-5907s (1995); anti-EpCAM antibodies such as 17-1A (PANOREX®); anti-GpIIb/IIIa antibodies such as abciximab or c7E3 Fab (REOPRO®); anti-RSV antibodies such as MEDI-493 (SYNAGIS®); anti-CMV antibodies such as PROTOVIR®; anti-HIV antibodies such as PRO542; anti-hepatitis antibodies such as the anti-Hep B antibody OSTAVIR®; anti-CA 125 antibody OvaRex; anti-idiotypic GD3 epitope antibody BEC2; anti-αvβ3 antibody VITAXIN®; anti-human renal cell carcinoma antibody such as ch-G250; ING-1; anti-human 17-1A antibody (3622W94); anti-human colorectal tumor antibody (A33); anti-human melanoma antibody R24 directed against GD3 ganglioside; anti-human squamous-cell carcinoma (SF-25); and anti-human leukocyte antigen (HLA) antibodies such as Smart ID10 and the anti-HLA DR antibody Oncolym (Lym-1).

Examples of antibodies which bind the CD20 antigen include: “C2B8” which is now called “Rituximab” (“RITUXAN®”) (U.S. Pat. No. 5,736,137, expressly incorporated herein by reference); the yttrium-[90]-labeled 2B8 murine antibody designated “Y2B8” or “Ibritumomab Tiuxetan” ZEVALIN® (U.S. Pat. No. 5,736,137, expressly incorporated herein by reference); murine IgG2a “B1, ” also called “Tositumomab,” optionally labeled with ¹³¹I to generate the “¹³¹I-B1” antibody (iodine I131 tositumomab, BEXXAR™) (U.S. Pat. No. 5,595,721, expressly incorporated herein by reference); murine monoclonal antibody “1F5” (Press et al. Blood 69(2):584-591 (1987)); murine 2H7 and chimeric 2H7 antibody (U.S. Pat. No. 5,677,180, expressly incorporated herein by reference); humanized 2H7, including “humanized 2H7 v16” (see below); huMax-CD20 (Genmab, Denmark); AME-133 (Applied Molecular Evolution); and monoclonal antibodies L27, G28-2, 93-1B3, B-C1 or NU-B2 available from the International Leukocyte Typing Workshop (Valentine et al., In: Leukocyte Typing III (McMichael, Ed., p. 440, Oxford University Press (1987)).

Examples of antibodies which bind the CD19 antigen include the anti-CD19 antibodies in Hekman et al. Cancer Immunol. Immunother. 32:364-372 (1991) and Vlasveld et al. Cancer Immunol. Immunother. 40:37-47 (1995); and the B4 antibody in Kiesel et al. Leukemia Research II, 12: 1119 (1987).

The terms “rituximab” or “RITUXAN®” herein refer to the genetically engineered chimeric murine/human monoclonal antibody directed against the CD20 antigen and designated “C2B8” in U.S. Pat. No. 5,736,137, expressly incorporated herein by reference. The antibody is an IgG₁ kappa immunoglobulin containing murine light and heavy chain variable region sequences and human constant region sequences. Rituximab has a binding affinity for the CD20 antigen of approximately 8.0 nM.

Purely for the purposes herein, “humanized 2H7 v16” refers to an antibody comprising the variable light and variable heavy sequences shown below.

Variable light-chain domain of hu2H7 v16:

| | |
|---|---|
| DIQMTQSPSSLSASVGDRVTITCRASSSVSYMHWYQ | (SEQ ID NO: 1) |
| QKPGKAPKPLIYAPSNLASGVPSRFSGSGSGTDFTL | |
| TISSLQPEDFATYYCQQWSFNPPTFGQGTKVEIKR | |

Variable heavy-chain domain of hu2H7 v16:

| | |
|---|---|
| EVQLVESGGGLVQPGGSLRLSCAASGYTFTSYNMHW | (SEQ ID NO: 2) |
| VRQAPGKGLEWVGAIYPGNGDTSYNQKFKGRFTISV | |
| DKSKNTLYLQMNSLRAEDTAVYYCARVVYYSNSYWY | |
| FDVWGQGTLVTVSS. | |

Preferably humanized 2H7 v16 comprises the light chain amino acid sequence:

| | |
|---|---|
| MGWSCIILFLVATATGVHSDIQMTQSPSSLSASVGD | (SEQ ID NO: 3) |
| RVTITCRASSSVSYMHWYQQKPGKAPKPLIYAPSNL | |
| ASGVPSRFSGSGSGTDFTLTISSLQPEDFATYYCQQ | |
| WSFNPPTFGQGTKVEIKRTVAAPSVFIFPPSDEQLK | |
| SGTASVVCLLNNFYPREAKVQWKVDNALQSGNSQES | |
| VTEQDSKDSTYSLSSTLTLSKADYEKHKVYACEVTH | |
| QGLSSPVTKSFNRGEC; | |

and heavy chain amino acid sequence:

Substantial modifications in the biological properties of the antagonist or antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class.

Any cysteine residue not involved in maintaining the proper conformation of the antagonist or antibody also may be substituted, generally with serine, to improve the oxidative stability of the molecule and prevent aberrant crosslinking. Conversely, cysteine bond(s) may be added to the antagonist or antibody to improve its stability (particularly where the antagonist is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or in additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the antagonist or antibody alters the original glycosylation pattern of the antagonist or antibody. By altering is meant deleting one or more carbohydrate moieties found in the antagonist or antibody, and/or adding one or more glycosylation sites that are not present in the antagonist or antibody.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antagonist or antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antagonist or antibody (for O-linked glycosylation sites).

Antibodies with altered Fc region glycosylation are described in WO02/079255 (Reff and Davies) and WO 03/035835 (Presta), expressly incorporated herein by reference.

Nucleic acid molecules encoding amino acid sequence variants of the antagonist or antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antagonist or antibody.

It may be desirable to modify the antagonist or antibody of the invention with respect to effector function, e.g. so as to enhance antibody-dependent cell-mediated cytotoxicity (ADCC) and/or complement dependent cytotoxicity (CDC) of the antagonist or antibody. This may be achieved by introducing one or more amino acid substitutions in an Fc region of an antibody antagonist. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cell-mediated cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989). Antibodies with altered (increased or diminished) C1q binding and or CDC activity are described in U.S. Pat. Nos. 6,194,551B1 and 6,538,124B1 (Idusogie et al.), expressly incorporated herein by reference. Antibodies with altered (increased or diminished) FcR binding and/or ADCC activity are described in WO00/42072 (Presta, L.), expressly incorporated herein by reference.

To increase the serum half life of the antagonist or antibody, one may incorporate a salvage receptor binding epitope into the antagonist or antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule. Alternatively, or additionally, one may increase, or decrease, serum half-life by altering the amino acid sequence of the Fc region of an antibody to generate variants with altered FcRn binding. Antibodies with altered FcRn binding and/or serum half life are described in WO00/42072 (Presta, L.), expressly incorporated herein by reference.

V. Pharmaceutical Formulations

Therapeutic formulations of the antagonists or antibodies used in accordance with the present invention are prepared for storage by mixing an antagonist or antibody having the desired degree of purity with optional pharmaceutically acceptable carriers, excipients or stabilizers (Remington's Phannaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Exemplary CD20 antibody formulations are described in WO98/56418, expressly incorporated herein by reference. This publication describes a liquid multidose formulation comprising 40 mg/mL rituximab, 25 mM acetate, 150 mM trehalose, 0.9% benzyl alcohol, 0.02% polysorbate 20 at pH 5.0 that has a minimum shelf life of two years storage at 2-8° C. Another CD20 formulation of interest comprises 10 mg/mL rituximab in 9.0 mg/mL sodium chloride, 7.35 mg/mL sodium citrate dihydrate, 0.7 mg/mL polysorbate 80, and Sterile Water for Injection, pH 6.5.

Lyophilized formulations adapted for subcutaneous administration are described in WO97/04801 and U.S. Pat. No. 6,267,958 (Andya et al.). Such lyophilized formulations may be reconstituted with a suitable diluent to a high protein concentration and the reconstituted formulation may be administered subcutaneously to the mammal to be treated herein.

The formulation herein may also contain more than one active compound as necessary for the particular indication being treated, preferably those with complementary activities that do not adversely affect each other. For example, it may be desirable to further provide a cytotoxic agent, chemotherapeutic agent, cytokine or immunosuppressive agent. The effective amount of such other agents depends on the amount of antagonist or antibody present in the formulation, the type of disease or disorder or treatment, and other factors discussed above. These are generally used in the same dosages and with administration routes as used hereinbefore or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antagonist or antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

VI. Treatment with the Antagonist or Antibody

The present invention contemplates therapy of various diseases and disorders with antibodies and antagonists. Where the antibody or antagonist binds to a B cell surface marker, such as CD20, conditions to be treated include B cell malignancies (see U.S. Pat. No. 6,455,043B1, Grillo-Lopez, expressly incorporated herein by reference), and autoimmune diseases (see WO00/67796, Curd et al., expressly incorporated herein by reference). The antagonist or antibody which binds to a B cell surface marker may also be used to block an immune response to a foreign antigen, e.g. where the foreign antigen is an immunogenic drug or transplant (see WO01/03734, Grillo-Lopez et al., expressly incorporated herein by reference).

For the various indications disclosed herein, a composition comprising the antagonist or antibody will be formulated, dosed, and administered in a fashion consistent with good medical practice. Factors for consideration in this context include the particular disease or condition being treated, the particular mammal being treated, the clinical condition of the individual patient, the cause of the disease or condition, the site of delivery of the agent, the method of administration, the scheduling of administration, and other factors known to medical practitioners. The therapeutically effective amount of the antagonist or antibody to be administered will be governed by such considerations.

As a general proposition, the therapeutically effective amount of the antagonist or antibody administered parenterally per dose will be in the range of about 0.1 to 20 mg/kg of patient body weight per day, with the typical initial range of antagonist or antibody used being in the range of about 2 to 10 mg/kg.

The preferred antagonist is an antibody, e.g. an antibody such as Rituximab or humanized 2H7, which is not conjugated to a cytotoxic agent. Suitable dosages for an unconjugated antibody are, for example, in the range from about 20 mg/m² to about 1000 mg/m². In one embodiment, the dosage of the antibody differs from that presently recommended for Rituximab. Exemplary dosage regimens for the CD20 antibody include 375 mg/m2 weekly×4 or 8; or 1000 mg×2 (e.g. on days 1 and 15).

As noted above, however, these suggested amounts of antagonist or antibody are subject to a great deal of therapeutic discretion. The key factor in selecting an appropriate dose and scheduling is the result obtained, as indicated above. For example, relatively higher doses may be needed initially for the treatment of ongoing and acute diseases. To obtain the most efficacious results, depending on the disease or disorder, the antagonist or antibody is administered as close to the first sign, diagnosis, appearance, or occurrence of the disease or disorder as possible or during remissions of the disease or disorder.

The antagonist or antibody is administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration. In addition, the antagonist or antibody may suitably be administered by pulse infusion, e.g., with declining doses of the antagonist or antibody. Preferably the dosing is given by injections, most preferably intravenous or subcutaneous injections, depending in part on whether the administration is brief or chronic.

One may administer other compounds, such as cytotoxic agents, chemotherapeutic agents, immunosuppressive agents and/or cytokines with the antagonists or antibodies herein. The combined administration includes coadministration, using separate formulations or a single pharmaceutical formulation, and consecutive administration in either order, wherein preferably there is a time period while both (or all) active agents simultaneously exert their biological activities.

For RA, and other autoimmune diseases, the antagonist or antibody (e.g. a CD20 antibody) may be combined with any one or more of the immunosuppressive agents, chemotherapeutic agents and/or cytokines listed in the definitions section above; any one or more disease-modifying antirheumatic drugs (DMARDs), such as hydroxycloroquine, sulfasalazine, methotrexate, leflunomide, azathioprine, D-penicillamine, Gold (oral), Gold (intramuscular), minocycline, cyclosporine, Staphylococcal protein A immunoadsorption; intravenous immunoglobulin (IVIG); nonsteroidal antiinflammatory drugs (NSAIDs); glucocorticoid (e.g. via joint injection); corticosteroid (e.g. methylprednisolone and/or prednisone); folate; an anti-tumor necrosis factor (TNF) antibody, e.g. etanercept/ENBREL™, infliximab/REMICADE™, D2E7 (Knoll) or CDP-870 (Celltech); IL-1R antagonist (e.g. Kineret); 1L-10 antagonist (e.g. Ilodecakin); a blood clotting modulator (e.g. WinRho); an IL-6 antagonist/anti-TNF (CBP 1011); CD40 antagonist (e.g. IDEC 131); Ig-Fc receptor antagonist (MDX33); immunomodulator (e.g. thalidomide or ImmuDyn); anti-CD5 antibody (e.g. H5g1.1); macrophage inhibitor (e.g. MDX 33); costimulatory blocker (e.g. BMS 188667 or Tolerimab); complement inhibitor (e.g. h5G1.1, 3E10 or an anti-decay accelerating factor (DAF) antibody); or IL-2 antagonist (zxSMART).

For B cell malignancies, the antagonist or antibody (e.g. a CD20 antibody) may be combined with a chemotherapeutic agent; cytokine, e.g. a lymphokine such as IL-2, IL-12, or an interferon, such as interferon alpha-2a; other antibody, e.g., a radiolabeled antibody such as ibritumomab tiuxetan (ZEVALIN®), iodine I¹³¹ tositumomab (BEXXAR™), ¹³¹I Lym-1 (ONCOLYM™), ⁹⁰Y-LYMPHOCIDE™; anti-CD52 antibody, such as alemtuzumab (CAMPATH-1H™), anti-HLA-DR-β antibody, such as apolizumab, anti-CD80 antibody (e.g. IDEC-114), epratuzumab, Hu1D10 (SMART 1D10™), CD19 antibody, CD40 antibody or CD22 antibody; an immunomodulator (e.g. thalidomide or ImmuDyn); an inhibitor of angiogenesis (e.g. an anti-vascular endothelial growth factor (VEGF) antibody such as AVASTIN™ or thalidomide); idiotype vaccine (EPOCH); ONCO-TCS™; HSPPC-96 (ONCOPHAGE™); liposomal therapy (e.g. daunorubicin citrate liposome), etc.

The preferred chemotherapy agents for combining with a CD20 antibody (or antagonist that binds to a B cell surface marker) are alkylator or anthracycline-based chemotherapeutic agents or fludarabine-based chemotherapeutic agents; cisplatin, fludarabine, vinblastine, doxorubicin, cyclophosphamide, and/or vincristine. With respect to CD20 antibodies or other antibodies that bind to a B cell surface marker, particularly desirable chemotherapies for combining with the antibody include, but are not limited to: cyclophosphamide, doxorubicin, vincristine and prednisone (CHOP) (Czuczman et al. J Clin Oncol 17:268-76 (1999)); cyclophosphamide, vincristine, and prednisone (CVP); fludarabine (e.g. for treating CLL); fludarabine, cyclophosphamide, and mitoxantrone (FCM); or doxorubicin, bleomycin, vinblastine, and dacarbazine (ABVD).

The antagonist or antibody may also be used in myeloablative regimens. For instance, the antagonist or antibody may be used for in vivo purging prior to stem cell collection, or post-transplantation, for eradication of minimal residual disease.

Aside from administration of protein antagonists to the patient the present application contemplates administration of antagonists or antibodies by gene therapy. See, for example, WO96/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antagonist or antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. USA 87:3410-3414 (1990). For review of the currently known gene marking and gene therapy protocols see Anderson et al., Science 256:808-813 (1992). See also WO 93/25673 and the references cited therein.

Further details of the invention are illustrated by the following non-limiting Examples. The disclosures of all citations in the specification are expressly incorporated herein by reference.

### EXAMPLE 1

### A Complement-Dependent Cytotoxicity Assay for Detecting Neutralizing Antibodies Against Rituximab

Rituximab exerts its biological function by depleting CD20+B cells through antibody-dependent cell-mediated cytotoxicity (ADCC), complement-dependent cytotoxicity (CDC), or both. In vitro, the CDC activity can be measured by incubating CD20+ WIL2-S lymphoma cells with human complement in the absence or presence of different concentrations of Rituximab. Cytotoxicity is then measured by quantifying live cells using ALAMAR BLUE® (Gazzano-Santoro et al., J. Immunol. Methods 202 163-171 (1997)).

In this example, serum samples from patients treated with Rituximab which resulted in HACA were identified. HACA positive serum, which was confirmed by immunodepletion, was then subjected to the neutralizing antibody assay described below. The HACA assay is a bridging format with Rituximab as the capture reagent and biotinylated Rituximab as the detection reagent. The assay has a calibrated standard curve prepared with polyclonal goat antibodies to Rituximab. The minimum dilution of a sample in the assay is 1/5, with the lowest standard at 1 RU (relative unit)/mL. A sample response below 5RU/mL (value corrected for 1/5 dilution factor) is considered negative for HACA.

An assay for detecting neutralizing antibodies against Rituximab was developed. The neutralizing antibody assay was performed using RPMI 1640 culture medium supplemented with 0.1% bovine serum albumin (BSA), 20 mM HEPES (pH 7.2-7.4), and 0.1 mM gentamicin. The assay was developed and calibrated using affinity purified polyclonal goat antibodies to Rituximab. When assay was performed in buffer matrix, typically 1-10 μL of goat anti-Rituximab was preincubated with 50 μL of various concentrations of Rituximab (0-10 μg/mL) in a flat-bottomed 96-well tissue culture plate. After preincubation at room temperature for 1-2 hours, 50 μL of a 1/3 human complement diluted in assay medium, 50 μL of WIL2-S lymphoblast cells of 10⁶ cells/mL suspended in assay medium were added, and the mixture was incubated for 2 hours at 37° C. and 5% CO₂ to facilitate complement-mediated cell lysis. 50 μL of undiluted AlamarBlue™ was then added and the incubation continued for 15-26 hours. The plates were allowed to cool to room temperature for 10 minutes by shaking and the fluorescence was read using a 96-well fluorometer with excitation at 530 nm and emission at 590 nm. Relative fluorescence units (RFU) were plotted against Rituximab concentration using a 4-parameter curve-fitting program (Softmax). By comparing the two curves with and without antibody preincubation, the neutralizing ability of anti-Rituximab antibodies can be determined. If the anti-Rituximab antibodies neutralized twenty percent or greater activity of Rituximab at a given concentration, the anti-Rituximab was defined as positive for neutralizing capability. This could be further quantified by determining the amount of anti-Rituximab to neutralize 1 μg of Rituximab. It was determined that the molar ratio for the goat anti-Rituximab polyclonal antibodies to neutralize Rituximab is approximately 3 to 1.

Since most patient samples for testing are serum samples, the serum matrix effect on assay performance was evaluated. Inclusion of 5% and 10% normal human serum in the assay medium had minimum effect on a 4-parameter fit curve. Signal suppression of upper asymptote was observed when serum concentration was above 20%. However, serum could be tolerated up to 50% without a significant shift in IC₅₀ values. These data demonstrated the feasibility of using CDC assay for detecting anti-Rituximab antibodies without further manipulating patient's serum samples. When testing serum samples, up to 50 μL of serum was incubated with 50 μL of Rituximab dilutions before complement and cell suspension addition. The rest of the procedures were the same as described above. For data analysis, the neutralizing ability of Rituximab-treated serum was compared individually with pre-treatment serum to determine neutralizing activity. The sensitivity/limit of detection of the assay in serum matrix was determined by spiking affinity purified goat anti-Rituximab into normal human serum. Using the current assay format, the lowest neutralizing antibody amount in serum that can be detected is approximately 1 μg/mL.

Rituximab treated systemic lupus erythematosus (SLE) patient samples with an antibody response (HACA+) by the ELISA assay above were tested in the neutralizing antibody assay. Significant differences were observed between baseline serum and serum following Rituximab treatment. The CDC activity was either completely or partially blocked with HACA+ sera, indicating neutralizing activities in the treated samples. In comparison, serum samples obtained prior to Rituximab treatment showed no neutralizing activity.

In summary, the Example describes a cell-based functional assay, complement-dependent cytotoxicity (CDC) assay, for detecting neutralizing activity in the serum of Rituximab treated patients. This assay will help largely in characterizing the nature of an anti-drug antibody response; therefore it will be of great value when evaluating drug safety and efficacy.

### EXAMPLE 2

### Therapy of Autoimmune Disease

According to one embodiment of the invention herein, the assay described herein may be used in relation to a treatment regimen for patients with an autoimmune disease. Exemplary autoimmune diseases include rheumatoid arthritis (RA), including juvenile rheumatoid arthritis, systemic lupus erythematosus (SLE), including lupus nephritis, Wegener's disease, inflammatory bowel disease, idiopathic or immune thrombocytopenic purpura (ITP), thrombotic thrombocytopenic purpura (TTP), autoimmune thrombocytopenia, multiple sclerosis (MS), psoriasis, IgA nephropathy, IgM polyneuropathies, myasthenia gravis, vasculitis, diabetes mellitus, Reynaud's syndrome, Sjogren's syndrome, glomerulonephritis, autoimmune hemolytic anemia, etc.

An antibody that binds CD20 (e.g. Rituximab or humanized 2H7) is administered to the patient in an amount effective to treat the autoimmune disease in question. For instance, the antibody may be dosed at 375 mg/m² every week for 4 or 8 weeks, or 1000 mg on Days 1 and 15. The antibody is optionally combined with one or more other drugs that treat the autoimmune disease, such as immunosuppressive agents, chemotherapeutic agents and/or cytokines listed in the definitions section above; any one or more of disease-modifying antirheumatic drugs (DMARDs) such as hydroxycloroquine, sulfasalazine, methotrexate, leflunomide, azathioprine, D-penicillamine, Gold (oral), Gold (intramuscular), minocycline, cyclosporine, Staphylococcal protein A immunoadsorption; intravenous immunoglobulin (IVIG); nonsteroidal antiinflammatory drugs (NSAIDs); glucocorticoid (e.g. via joint injection); corticosteroid (e.g. methylprednisolone and/or prednisone); folate; an anti-tumor necrosis factor (TNF) antibody, e.g. etanercept/ENBREL™, infliximab/REMICADE™, D2E7 (Knoll) or CDP-870 (Celltech); IL-1R antagonist (e.g. Kineret); 1L-10 antagonist (e.g. Ilodecakin); a blood clotting modulator (e.g. WinRho); an IL-6 antagonist/anti-TNF (CBP 1011); CD40 antagonist (e.g. IDEC 131); Ig-Fc receptor antagonist (MDX33); immunomodulator (e.g. thalidomide or ImmuDyn); anti-CD5 antibody (e.g. H5g1.1); macrophage inhibitor (e.g. MDX 33); costimulatory blocker (e.g. BMS 188667 or Tolerimab); complement inhibitor (e.g. h5G 1.1, 3E10 or an anti-decay accelerating factor (DAF) antibody); or IL-2 antagonist (zxSMART).

A biological sample of serum, which may comprise HACA (directed against Rituximab) or HAHA (directed against humanized 2H7), is obtained from the patient at baseline, and 3, 6 and 9 months. The serum is subjected to an ELISA to determine whether HACA or HAHA is present therein. The assay is described in Example 1 above.

Serum which is demonstrated to contain HACA or HAHA is then tested for neutralizing antibodies as in Example 1 above. In comparison to the same amount of pre-treatment counterpart (i.e., HACA and HAHA negative), a sample neutralizing about 20% or greater activity of Rituximab or humanized 2H7 at a given concentration, may be considered positive for neutralizing antibody directed against Rituximab or humanized 2H7. A positive result is indicative of reduced efficacy of the antibody in treating the autoimmune disease.

### EXAMPLE 3

### Therapy of B cell Malignancy

A patient with a CD20 positive B cell malignancy, such as Hodgkin's disease including lymphocyte predominant Hodgkin's disease (LPHD), non-Hodgkin's lymphoma (NHL), follicular center cell (FCC) lymphoma, acute lymphocytic leukemia (ALL), chronic lymphocytic leukemia (CLL), hairy cell leukemia, plasmacytoid lymphocytic lymphoma, mantle cell lymphoma, AIDS or HIV-related lymphoma, multiple myeloma, central nervous system (CNS) lymphoma, post-transplant lymphoproliferative disorder (PTLD), Waldenstrom's macroglobulinemia (lymphoplasmacytic lymphoma), mucosa-associated lymphoid tissue (MALT) lymphoma, or marginal zone lymphoma/leukemia, is treated according to this example.

An antibody that binds CD20 (e.g. Rituximab or humanized 2H7) is administered to the patient in an amount effective to treat the B cell malignancy in question. For instance, the antibody may be dosed at 375 mg/m² every week for 4 or 8 weeks.

Optionally, the CD20 antibody is combined with one or more chemotherapeutic agents. The preferred chemotherapy agents for combining with a CD20 antibody are alkylator or anthracycline-based chemotherapeutic agents or fludarabine-based chemotherapeutic agents; cisplatin, fludarabine, vinblastine, doxorubicin, cyclophosphamide, and/or vincristine. Particularly desirable chemotherapies for combining with the antibody include, but are not limited to: cyclophosphamide, doxorubicin, vincristine and prednisone (CHOP) (Czuczman et al. J Clin Oncol 17:268-76 (1999)); cyclophosphamide, vincristine, and prednisone (CVP); fludarabine (e.g. for treating CLL); fludarabine, cyclophosphamide, and mitoxantrone (FCM); or doxorubicin, bleomycin, vinblastine, and dacarbazine (ABVD) etc.

A biological sample of serum, which may contain HACA (directed against Rituximab) or HAHA (directed against humanized 2H7), is obtained from the patient at baseline, and 3, 6 and 9 months. The serum is subjected to an ELISA to determine whether HACA or HAHA is present therein. The assay is described in Example 1 above.

Serum which is demonstrated to contain HACA or HAHA is then tested for neutralizing antibodies as in Example 1 above. In comparison to the same amount of pre-treatment counterpart (i.e., HACA and HAHA negative), a sample neutralizing about 20% or greater activity of Rituximab or humanized 2H7 at a given concentration, may be considered positive for neutralizing antibody directed against Rituximab or humanized 2H7. The presence of neutralizing antibodies indicates reduced effectiveness of the antibody in treating the B cell malignancy.

### EXAMPLE 4

### Blocking an Immune Response to a Foreign Antigen

In the present example, an anti-CD20 antibody is used to block an immune response to a foreign antigen such as a therapeutic protein (e.g. a murine antibody or an immunotoxin), gene therapy viral vector, blood factor (e.g. Factor VIII), platelets, or transplant etc.

A suitable dosage of the CD20 antibody is 375 mg/m² by four or eight infusions given every week. Administration of the CD20 antibody will reduce or eliminate an immune response in the patients, and thereby facilitate successful therapy.

For blocking an immune response to a transplant, the CD20 antibody may be used as part of combination immunosuppressive regimens for prophylaxis of acute rejection. In this setting, a CD20 antibody, such as Rituximab or humanized 2H7, is administered in the peri-transplant period as part of a sequential combination regimen that includes T cell directed agents such as cyclosporine, corticosteroids, mycophenolate mofetil, with or without an anti-IL2 receptor antibody. Hence, the CD20 antibody would be considered part of an induction regimen, to be used in conjunction with chronic immunosuppressive therapies. The CD20 antibody may contribute to prevention of an allorejection response by inhibiting alloantibody production and/or affecting alloantigen presentation through depletion of antigen-presenting cells.

Dosages of the further immunosuppressive agents are as follows: cyclosporine (5 mg/kg/day); corticosteroids (1 mg/kg, gradually tapered off); mycophenolate mofetil (1 gram given twice a day); and anti-IL2 receptor antibody (1 mg/kg, five infusions given weekly). The CD20 antibody may also be combined with other induction immunosuppressive drugs, such as polyclonal anti-lymphocyte antibodies or monoclonal anti-CD3 antibodies; maintenance immunosuppressive drugs, such as calcineurin inhibitors (e.g., tacrolimus) and antiproliferative agents (such as azathioprine, leflunomide or sirolimus); or combination regimens that include blockade of T cell costimulation, blockade of T cell adhesion molecules of blockade of T cell accessory molecules.

Aside from prophylaxis of acute rejection, CD20 antibodies may be used to treat acute rejection. Suitable dosages of the CD20 are as described above. The CD20 antibody is optionally combined with a CD3 monoclonal antibody and/or corticosteroids in the treatment of acute rejection.

CD20 antibodies may also be used (a) later in the post-transplant period alone, or in combination with other immunosuppressive agents and/or costimulatory blockade, for treatment or prophylaxis of “chronic” allograft rejection; (b) as part of a tolerance-inducing regimen; or (c) in the setting of xenotransplantation.

A biological sample of serum, which may contain HACA (directed against Rituximab) or HAHA (directed against humanized 2H7), is obtained from the patient at baseline, and 3, 6 and 9 months. The serum is subjected to an ELISA to determine whether HACA or HAHA is present therein. The assay is described in Example 1 above.

Serum which is demonstrated to contain HACA or HAHA is then tested for neutralizing antibodies as in Example 1 above. In comparison to the same amount of pre-treatment counterpart (i.e., HACA and HAHA negative), a sample neutralizing about 20% or greater activity of Rituximab or humanized 2H7 at a given concentration, may be considered positive for neutralizing antibody directed against Rituximab or humanized 2H7. Where a neutralizing antibody response is detected, this indicates the antibody has reduced ability to block an immune response to the foreign antigen in question.

1. A method for evaluating the efficacy of an antibody that binds CD20 comprising measuring the ability of a biological sample from a patient treated with the CD20 antibody to block a biological activity of the CD20 antibody.

2. The method of claim 1 wherein the biological activity is selected from the group consisting of complement-dependent cytotoxicity (CDC), antibody-dependent cell-mediated cytotoxicity (ADCC), apoptosis, and inhibition of cell growth.

3. The method of claim 1 wherein the biological activity is complement-dependent cytotoxicity (CDC).

4. The method of claim 1 wherein the CD20 antibody is rituximab.

5. The method of claim 1 wherein the CD20 antibody is humanized 2H7.

6. The method of claim 1 wherein the biological sample comprises antibodies from the patient that bind the CD20 antibody.

7. The method of claim 6 wherein the biological sample has been subjected to an assay that determines the presence of antibodies from the patient that bind the CD20 antibody in a biological sample from the patient.

8. The method of claim 1 wherein the biological sample comprises serum from the patient.

9. The method of claim 1 wherein the patient has an autoimmune disease.

10. The method of claim 9 wherein the autoimmune disease is selected from the group consisting of rheumatoid arthritis (RA), systemic lupus erythematosus (SLE), Wegener's disease, inflammatory bowel disease, idiopathic or immune thrombocytopenic purpura (ITP), thrombotic thrombocytopenic purpura (TTP), autoimmune thrombocytopenia, multiple sclerosis (MS), psoriasis, IgA nephropathy, IgM polyneuropathies, myasthenia gravis, vasculitis, diabetes mellitus, Reynaud's syndrome, Sjogren's syndrome, glomerulonephritis, and autoimmune hemolytic anemia.

11. The method of claim 1 wherein the patient has a B cell malignancy.

12. The method of claim 11 wherein the B cell malignancy is selected from the group consisting of Hodgkin's disease, non-Hodgkin's lymphoma (NHL), follicular center cell (FCC) lymphoma, acute lymphocytic leukemia (ALL), chronic lymphocytic leukemia (CLL), hairy cell leukemia, plasmacytoid lymphocytic lymphoma, mantle cell lymphoma, AIDS or HIV-related lymphoma, multiple myeloma, central nervous system (CNS) lymphoma, post-transplant lymphoproliferative disorder (PTLD), Waldenstrom's, mucosa-associated lymphoid tissue (MALT) lymphoma, and marginal zone lymphoma/leukemia.

13. The method of claim 1 wherein the patient was treated with the CD20 antibody to block an immune response to a foreign antigen.

14. The method of claim 13 wherein the foreign antigen comprises a therapeutic agent.

15. The method of claim 13 wherein the foreign antigen is selected from the group consisting of an antibody, a toxin, a gene therapy viral vector, a graft, an infectious agent, and an alloantigen.

16. The method of claim 13 wherein the foreign antigen is a graft.

17. The method of claim 3 wherein the assay comprises exposing CD20 positive cells to complement in the presence of the CD20 antibody and the biological sample and then determining viability of the exposed cells.

18. A method of immunotherapy comprising
administering an antibody that binds CD20 to a patient; and
measuring the ability of a biological sample from the patient to block a biological activity of the CD20 antibody.

19. A method of detecting neutralizing antibodies to a therapeutic antibody comprising
exposing cells that express an antigen to which the therapeutic antibody binds to complement in the presence of the therapeutic antibody and a biological sample from a patient treated therewith; and
determining complement-dependent cytotoxicity (CDC) activity of the therapeutic antibody, wherein a reduction in the CDC activity indicates the presence of neutralizing antibodies in the biological sample.

20. A method of evaluating the efficacy of an antagonist that binds a B cell surface marker comprising measuring the ability of a biological sample from a patient treated with the antagonist to block a biological activity of the antagonist.

21. A method of immunotherapy comprising
administering an antibody that binds a B cell surface marker to a patient; and
measuring the ability of a biological sample from the patient to block a biological activity of the antibody.
